(19) United States
(12) Patent Application Publication
(10) Pub. No.: US 2007/0014741 A1
(43) Pub. Date: Jan. 18, 2007

(54) Collagen eucalyptus toothpaste

(76) Inventor: Chih-Yung Chiu, Taipei (TW)

Correspondence Address: LAW OFFICES OF CLEMENT CHENG, 17220 NEWHOPE STREET #127, FOUNTAIN VALLEY, CA, 92708, US

(21) Appl. No.: 11/182,428

(22) Filed: Jul. 16, 2005

(51) Int. Cl. A61K 8/96; A61K 8/21; A61K 8/97

(52) U.S. Cl. 424/50; 424/58; 424/52

(57) ABSTRACT

A toothpaste, dental cream, or mouth spray composition for soothing the gums and teeth has base ingredients such as sorbitol, hydrated silica, polyethylene glycol and water; and a combination of eucalyptus oil and collagen, being present at less than about 10% by weight.

### DISCUSSION OF RELATED ART

Different formulations of toothpaste provide a wide range of benefits. For example, weight-loss toothpaste as seen in U.S. Pat. No. 6,610,277 formulated by Zuckerman provides a combination of appetite suppressants comprising effective amounts of Griffonia Simplicifolia, Gymnema Sylvestre, and Citrus Aurantium, being present at about 5.5% to about 22% by weight concentrations. Toothpaste can cure skeletal diseases as seen in U.S. Pat. No. 6,911,425 to Kumagai which uses peptide sequences of amino acids having integrin binding motif, glycosaminoglycan binding motif, and a calcium binding motif, with the remainder of amino acids contiguous with the RGD sequence in matrix extracellular phosphoglycoprotein. The toothpaste enhances bone growth and reduces excessive urinary phosphate loss. An anti ‘orange juice effect’ toothpaste has also been formulated. Chang in U.S. Pat. No. 5,120,528 provides a sodium lauryl sulfate free toothpaste with protein fatty acid condensates and acid hydrolyzed animal proteins, together with a flavor oil, a high foaming sulfosuccinate and a natural emulsifier. Toothpaste can also provide an herbal remedy.

Toothpaste has standard formulations for the base as described in U.S. Pat. No. 6,610,277 to Zuckerman the disclosure of which is incorporated herein by reference.
“The standard toothpaste base of the instant invention preferably comprises substantially from 10-30% Vegetable Glycerin; substantially from 10-30% Sorbitol; substantially from 10-30% Hydrated Silica; substantially from 10-30% Purified Water; substantially from 10-30% Xylitol; substantially from 1-3% Carrageenan; substantially from 1-3% Sodium Lauryl Sulfate; substantially from 1-3% Titanium Dioxide; and substantially from 0.30-1% Oil of Wintergreen. Sorbitol is the major humectant ingredient because of its sweet taste. Minimal amounts of polyethylene are used due to its bitter taste. A mixture of sorbitol and glycerin is preferred. In the toothpaste, dental gel or dental cream, the humectant constitutes about 65-75% by weight of the composition and the water content is about 10-30% by weight of the toothpaste.
It is preferred to use a gelling agent in dental creams or gels, such as the natural and synthetic gums and gum like materials, for example Carrageenan, Irish moss, gum tragacanth, cellulose gums such as methyl cellulose, carboxymethyl cellulose, hydroxyethyl cellulose, hydroxmethyl cellulose, polyvinylpyrrolidone, hydrophilic colloidal carboxyvinyl polymers, such as those sold under the trademark Carbopol 934 and 940 and synthetic silicated clays such as those sold under the trademark Laponite CP and Laponite SP. These grades of Laponite have the formula (Si.sub.8 Mg.sub.5 Li.sub.0.6 O.sub.24).sup.0.6 -Na.sup.0.6+. The gelling agent constitutes about 1.0-3.0% by weight of the toothpaste formulation.
The toothpaste formulations will generally also include a dentally acceptable, substantially water insoluble polishing agent of the type commonly employed in dental creams. Representative polishing agents include, for example, dicalcium phosphate, tricalcium phosphate, insoluble sodium metaphosphate, aluminum hydroxide, including hydrated alumina, colloidal silica, hydrated silica, precipitated silica and magnesium carbonate, calcium carbonate, calcium pyrophosphate, and bentonite, including suitable mixtures thereof. It is preferred to use silica-containing polishing agents such as amorphous hydrated silicon dioxide (SiO.sub.2 H.sub.2 O), known as Zeodent/Zeofree/Zeosyl/Zeothix obtainable from J.M. Huber Corporation, which is in the form of a white, odorless powder having an average particle size of 8-10 micrometers and a density of 2 g/ml. at 25.degree. C. Amorphous silica, also called silica gel and silicic acid, is also obtainable from W.R. Grace & Co. as Sylodent 704 which is a dry white powder having an average particle size of 4 microns and a specific gravity of 2.1. Sylox.RTM. is another amorphous silica provided by W.R. Grace & Co., in the form of a dry white powder having an average size of 1.5-12 microns. The preferred siliceous containing polishing agent constitutes about 10-30% by weight of the dental cream formulations.
When the toothpaste is a visually clear gel or opacified gel, a polishing agent of colloidal silica, such as those sold under the trademark Syloid as Syloid 72 and Syloid 74 or under the trademark Santocel as Santocel 100 and synthetic alkali metal aluminosilicate complexes (including silica containing combined alumina) may be particularly useful. They have refractive indices close to the refractive indices of gelling agents-liquid systems commonly used in toothpaste (which generally include humectants such as glycerine and sorbitol).
Organic surface-active agents are preferably used in the composition of the present invention to assist in achieving thorough and complete dispersion of the compositions of the present invention throughout the oral cavity and render the said compositions more cosmetically acceptable. The organic surface-active agent material may be anionic or nonionic, in nature, and it is preferred to employ as the surface-active agent a detersive material which imparts to the composition detersive and foaming properties. Suitable anionic surfactants include water-soluble salts of higher fatty acid monoglyceride monosulfates, such as the sodium salt of the monosulfated monoglyceride of hydrogenated coconut oil fatty acids, higher alkyl sulfates, such as sodium lauryl sulfate, alkyl aryl sulfonates, such as sodium dodecyl benzene sulfonate, higher alkyl sulfoacetates, higher fatty acid esters of 1,2-dihydroxy propane sulfonates, olefin sulfonates and the like.
Other particularly suitable surface active materials include nonionic agents such as condensates of sorbitan monostearate with approximately 20 moles of ethylene oxide; condensates of ethylene oxide with propylene oxide; condensates of propylene glycol (Pluronics); condensation products of an alpha-olefin oxide containing 10 to 20 carbon atoms; a polyhydric alcohol containing 2 to 10 carbons; and 2 to 6 hydroxyl groups and either ethylene oxide or a heteric mixture of ethylene oxide and propylene oxide. The resultant detergents are heteric polymers having a molecular weight in the range of 400 to 1600 and containing 40% to 80% by weight of ethylene oxide, with an alpha-olefin oxide to polyhydric alcohol mole ratio in the range of 1:1 to 1:3. The amount of anionic or nonionic surfacant constitutes about 1-3% by weight of the toothpaste formulation.
The toothpaste of this invention may also contain conventional additional ingredients such as coloring or whitening agents and preservatives. These additional ingredients may each be added to the toothpaste in minimal amounts of up to 5% by weight, and preferably up to 1 . . .”
“The toothpaste of this invention is prepared by conventional methods of making toothpaste and/or dental creams or dental gels. More specifically, the gelling agent such as a cellulose gum is dispersed in glycerine, to which is added an aqueous solution containing the sweetening agent such as xylitol, followed by the addition of sorbitol and mixing for a period of about 20 minutes to hydrate the gum, mixing the gum mixture with the polishing agent in a mixer under a vacuum of 28-30 inches of pressure. Lastly, the flavor, the surfactant and natural herbs are added to the vacuum mixer, mixed for a period of about 15 minutes, and the final mixture is placed in a tube.”

### DETAILED DESCRIPTION OF THE PREFERRED EMBODIMENT

The present invention formulation provides a holistic soothing toothpaste for the tongue, gums, and teeth. Collagen can be derived from culture or by animal cartilage such as chicken or other avian. The collagen is processed and mixed with the toothpaste base. A small amount of collagen is generally used, and is typically less than 5%. The collagen can be prepared from ground chicken cartilage so that the protein chain of approximately 1400 amino acids is not substantially broken or denatured.

The collagen is preferable medical grade so that it is above 80% pure. The present embodiments and best mode is to use 99% pure collagen. Typically, cosmetic grade collagen of approximately 50% purity is not recommended. Processing medical grade collagen is well-known in the art and need not be described here for operation of the invention.

The eucalyptus oil is also typically less than 5% by weight. The eucalyptus oil, preferably from Australian eucalyptus trees is also processed and mixed with the toothpaste base.

Citric acid can be used to adjust the pH of the formulation. The pH is adjusted so that it is approximately between about pH 8 to 10. Alternatively, collagen can be added to the base of the toothpaste formulation without the Eucalyptus oil. If so, the amount of the citric acid in the formulation is adjusted accordingly.

Best Mode Embodiment

The following is a best mode of the present invention.

| % by weight in | | |
|---|---|---|
| formula | INCI name | Scientific Name |
| 44% | Sorbitol | Sorbitol |
| 22.5% | Aqua | Water, filtered |
| 0.2% | Saccharin | Saccharin |
| 0.3% | Sodium Flouride | Sodium fluoride |
| 0.1% | Sodium Benzonate | Sodium benzonate |
| 1% | CI 77891 | Titanium dioxide |
| 5.5% | PEG-32 | Polyethylene glycol 1500 |
| 5% | Hydrated Silica | Thickening silica (Sorbosil TC 15/Sident 22S) |
| 3.5% | Hydrated Silica | Sorbisol AC43 |
| 14.5% | Hydrated Silica | Abrasive Silica (Sorbisol AC 35) |
| 0.7% | Cellulose Gum | Sodium carboxymethyl cellulose-9M Type |
| | | (Walocel 2000 PA) |
| 1.5% | Sodium Laurl Sulfate | Sodium Laurl Sulfate (Empicol LZPV/C) |
| 0.05% | Collagen | Collagen |
| 0.01% | Eucalyptus Oil | Oil of Eucalyptus (90/95% cineole)CAS No. |
| | | 800-46-4 |
| 1% | Flavor | Flavor (TP 7890) |
| 0.2% | Citric Acid | Citric Acid (to adjust pH) |

Second Embodiment

| % by weight in | | |
|---|---|---|
| formula | INCI name | Scientific Name |
| 10-30% | Sorbitol | Sorbitol |
| 10-30% | Aqua | Water, filtered |
| 0.2% | Saccharin | Saccharin |
| 0.3% | Sodium Flouride | Sodium fluoride |
| 0.1% | Sodium Benzonate | Sodium benzonate |
| 1% | CI 77891 | Titanium dioxide |
| 5.5% | PEG-32 | Polyethylene glycol 1500 |
| 5% | Hydrated Silica | Thickening silica (Sorbosil TC 15/Sident 22S) |
| 3.5% | Hydrated Silica | Sorbisol AC43 |
| 10-30% | Hydrated Silica | Abrasive Silica (Sorbisol AC 35) |
| 0.7% | Cellulose Gum | Sodium carboxymethyl cellulose-9M Type |
| | | (Walocel 2000 PA) |
| .5-4% | Sodium Laurl Sulfate | Sodium Laurl Sulfate (Empicol LZPV/C) |
| 0.03-5% | Collagen | Collagen |
| 0.01-5% | Eucalyptus Oil | Oil of Eucalyptus (90/95% cineole)CAS No. |
| | | 800-46-4 |
| 1% | Flavor | Flavor (TP 7890) |
| 0.2% | Citric Acid | Citric Acid (to adjust pH) |
| 1-30% | Xylitol | |

The International Nomenclature of Cosmetic Ingredients, abbreviated INCI, is a nomenclature system. Sodium fluoride (NaF) is the most popular active ingredient in toothpaste to prevent cavities and is practically equivalent to sodium monofluorophosphate (SMFP).

Formulation variations may include food grade surfactants substitution for sodium lauryl sulfate or polyoxyethylene sorbitan diiostearate surfactants. Dental polishing agents may also be varied. The flavor may be varied such as by adding one or more flavors such as wintergreen, spearmint, or strawberry.

The invention describes preferred embodiments, and numerous additional modifications and changes may become readily apparent to a person of ordinary skill in the art after reading this disclosure without departing from the spirit of the present invention. The scope of the invention is not limited by the details of the detailed embodiments shown above.

1. A toothpaste, dental cream, or mouth spray composition, comprising:
a) toothpaste, dental cream, or mouth spray base ingredients; and
b) a combination of eucalyptus oil and collagen, being present at less than about 10% by weight.

2. The toothpaste, dental cream, or mouth spray composition of claim 1, further comprising citric acid.

3. The toothpaste, dental cream, or mouth spray composition of claim 1, wherein the pH is from 8 to 10.

4. The toothpaste, dental cream, or mouth spray composition of claim 1, further comprising sodium fluoride being present and at less than 5% by weight.

5. A toothpaste, dental cream, or mouth spray composition, comprising:
a) toothpaste, dental cream, or mouth spray base ingredients; and
b) a combination of eucalyptus oil at 0.01% to 0.3% by weight and animal collagen, being present and at less than 5% by weight.

6. The toothpaste, dental cream, or mouth spray composition of claim 5, further comprising citric acid.

7. The toothpaste, dental cream, or mouth spray composition of claim 5, wherein the pH is from 8 to 10.

8. The toothpaste, dental cream, or mouth spray composition of claim 5, further comprising sodium fluoride being present and at less than 5% by weight.

9. A toothpaste, dental cream, or mouth spray composition, comprising:
a) toothpaste, dental cream, or mouth spray base ingredients; and
b) collagen, being present at less than 5% by weight.

10. The toothpaste, dental cream, or mouth spray composition of claim 9, further comprising citric acid.

11. The toothpaste, dental cream, or mouth spray composition of claim 9, wherein the pH is from 8 to 10.

12. The toothpaste, dental cream, or mouth spray composition of claim 9, further comprising sodium fluoride being present and at less than 5% by weight.

13. The toothpaste, dental cream, or mouth spray composition of claim 9, wherein the collagen is animal collagen.

14. The toothpaste, dental cream, or mouth spray composition of claim 9, wherein the collagen is avian collagen.

15. The toothpaste, dental cream, or mouth spray composition of claim 9, wherein the collagen is chicken cartilage collagen.
